(19) United States
(12) Patent Application Publication
(10) Pub. No.: US 2011/0178325 A1
(43) Pub. Date: Jul. 21, 2011

(54) Automatic Process for Synthesizing F-18-FDDNP

(75) Inventors: Kang-Wei Chang, Longtan Shiang (TW); Yen-Hung Tu, Longtan Shiang (TW); Shih-Ying Lee, Longtan Shiang (TW); Jenn-Tzong Chen, Longtan Shiang (TW); Chia-Chieh Chen, Longtan Shiang (TW); Wuu-Jyh Lin, Longtan Shiang (TW)

(73) Assignee: ATOMIC ENERGY COUNCIL-INSTITUTE OF NUCLEAR ENERGY RESEARCH, Taoyuan County (TW)

(21) Appl. No.: 12/689,198

(22) Filed: Jan. 18, 2010

(51) Int. Cl. C07C 255/34

(52) U.S. Cl. 558/403

(57) ABSTRACT

Disclosed is an automatic process for synthesizing F-18-FDDNP. The automatic process includes a preparation subroutine and a synthesis subroutine. The automatic process is efficient and simple. That is, it can be used to produce a large amount of chemicals in a short period of time. Furthermore, it reduces the risk of human exposure to radiation.

### FIELD OF THE INVENTION

The present invention relates to an automatic process for synthesizing F-18-FDDNP for use in magnetic resonance imaging whereby the risk of human exposure to radiation is reduced.

### DESCRIPTION OF THE RELATED ARTS

Because of rapidly changing lifestyle, increasing stress and aging population, more and more people suffer CNS diseases. Among others, schizophrenia, anxiety and depression classified as mental disorders, Alzheimer's disease classified as a neuron-degeneration disease and Parkinson's disease classified as an ataxia telangiectasia syndrome are often seen. These diseases affect the quality of our life and consume a lot of resources of our society.

Alzheimer's disease is a progressive brain degeneration disease and is also called “dementia.” Alzheimer's disease is most often seen and incurable. Because Alzheimer's disease is often seen in senior citizens, the risk of Alzheimer's disease grows with the age. For people more than 85 years old, the risk of Alzheimer's disease is 30% to 35%. The clinical manifestations of this progressive disease include degeneration in cognition, degeneration in behavior and degeneration in metal condition. There are specific changes in neuron-pathology, i.e., the shrinking of the cortex region of the brain, broad suclus and gyrus regions of the brain and the extension of the ventricles of the brain. The most important pathological indicators are neurofibrillary tangles and senile plaques.

There are about 50000 patents of Alzheimer's disease. Many tiny, round sediments or plaques are found in the brains of these patients. The protein that tangles like a spider's net is starch-like protein and the key to the study of the mechanism of Alzheimer's disease and the treatment of the Alzheimer's disease.

There are three sorts of biological indicators in the study of Alzheimer's disease, i.e., styrlbenzenes, aminonaphthalene and thioflavin-S. The styrlbenzenes includes X-34, ISB, BSB, SB13 and IMSB. The aminonaphthalene includes FDDNP and FENE. The thioflavin-S includes 6-OH-BTA-1, TZDM and IMPY. F-18 can be added to the FDDNP of the aminonaphthalene to form F-18-FDDNP for use in positron emission tomography. The UCLA Hospital, the U.S.A., is promoting F-18-FDDNP. F-18-FDDNP is at the first stage of clinical test. However, labeling is done manually, and human exposure to radiation is high.

The present invention is therefore intended to obviate or at least alleviate the problems encountered in prior art.

### SUMMARY OF THE INVENTION

It is an objective of the present invention to provide an efficient and simple process for automatically synthesizing F-18-FDDNP.

It is another objective of the present invention to provide a process for automatically synthesizing F-18-FDDNP whereby the risk of human exposure to radiation is reduced.

To achieve the foregoing objectives, the automatic process includes a preparation subroutine and a synthesis subroutine. The preparation subroutine includes three steps. Potassium carbonate solution is filled in a first bottle, acetonitrile in a second bottle, water in a third bottle, dichloromethane in a fourth bottle, 0.1N hydrochloride in a fifth bottle and a precursor, triflate, in a sixth bottle. Each of the bottles is washed with methanol. A C-18 column is washed with ethanol and water. Another silicone column is washed with dichloromethane. The synthesis subroutine includes eight steps. F-18 is provided. The activity and dose are calculated. The F-18 is filled in a labeled bottle and heated on a heating plate. The loss of the water in the labeled bottle is observed. When some of the water condenses on the column, there is vaporization in the labeled bottle. The acetonitrile is filled in the labeled bottle from the second bottle in three rounds. When there is almost no water in the labeled bottle, DMTEAN, a precursor of FDDNP, dissolved in acetonitrile is filled in the labeled bottle from the sixth bottle for SN2 reaction. The reaction mixture in the labeled bottle is cooled with helium. Then, the 0.1N hydrochloride is filled in the labeled bottle from the fifth bottle for hydrolysis. The product of the hydrolysis is directed through the C-18 column from the labeled bottle. Then, the labeled bottle is washed with the water from the third bottle. The product is washed out from the C-18 column with the dichloromethane from the fourth bottle. The final product of F-18-FDDNP is collected in a collection bottle. Waste liquid produced in the process is collected in a waste bottle. The average yield of the F-18-FDDNP is 41.19±5.23%. The average radiochemistry purity is higher than 90%.

Other objectives, advantages and features of the present invention will become apparent from the following description referring to the attached drawings.

### BRIEF DESCRIPTIONS OF THE DRAWINGS

The present invention will be described via the detailed illustration of the preferred embodiment referring to the drawings.

FIG.1 is a flow chart of an automatic process for synthesizing F-18-FDDNP according to the preferred embodiment of the present invention

FIG.2 is a block diagram of a system for running the automatic process shown in FIG. 1.

### DESCRIPTION OF THE PREFERRED EMBODIMENTS

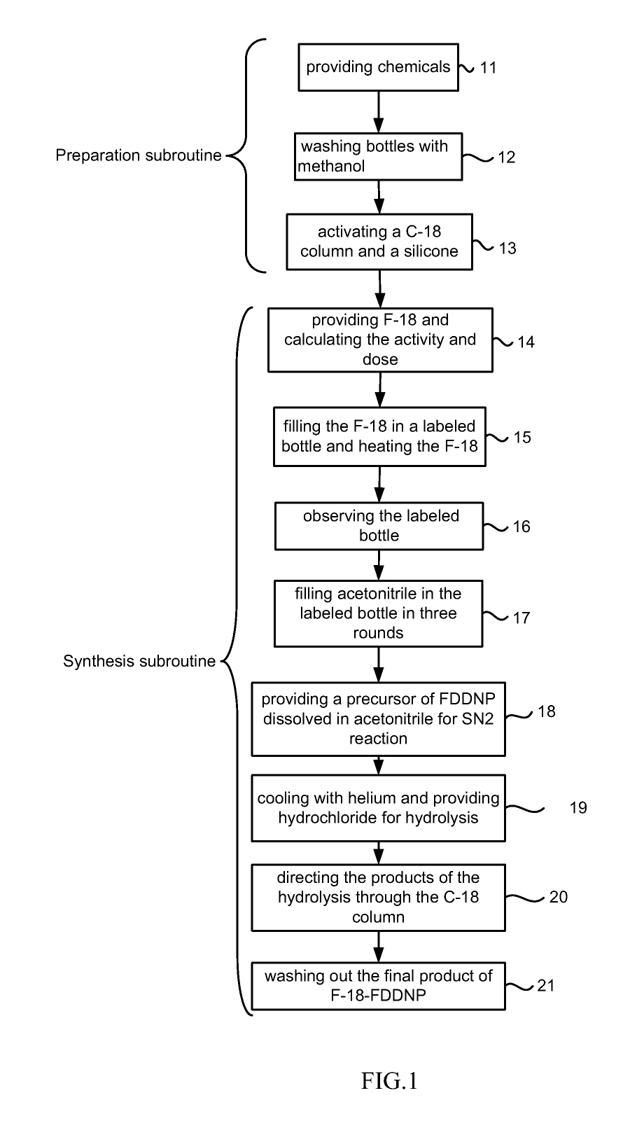

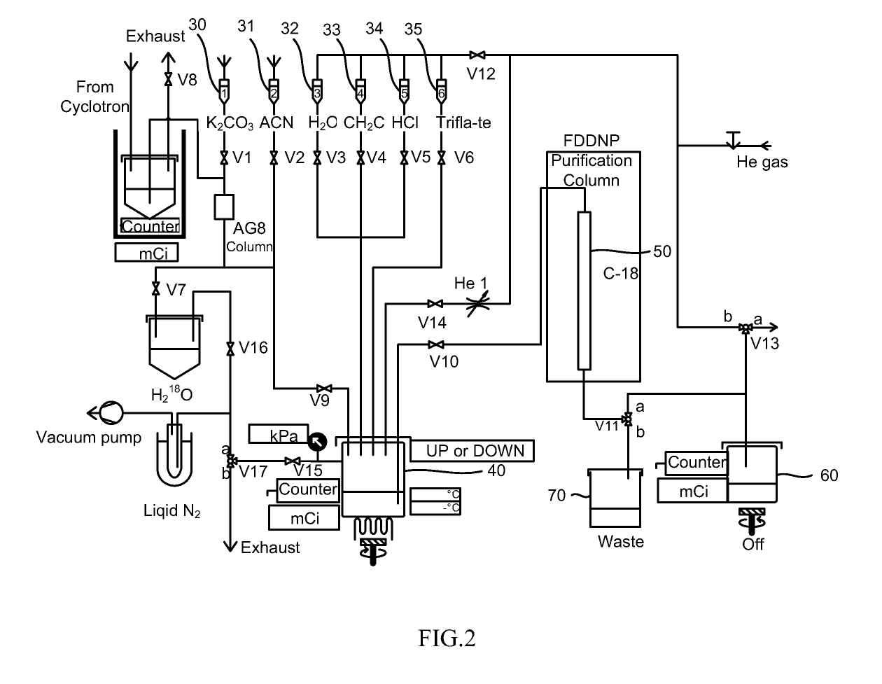

Referring to FIGS. 1 and 2, an automatic process for synthesizing F-18-FDDNP includes a preparation subroutine and a synthesis subroutine according to the preferred embodiment of the present invention.

The preparation subroutine includes three steps. At 11, chemicals are provided. In detail, potassium carbonate solution is filled in a first bottle 30, acetonitrile in a second bottle 31, water in a third bottle 32, dichloromethane in a fourth bottle 33, 0.1N hydrochloride in a fifth bottle 34 and a precursor, triflate, in a sixth bottle 35.

At 12, the bottles are washed. In detail, each of the bottles 30 through 35 is washed with methanol and dried.

At 13, a C-18 column 50 is washed with 5 ml of ethanol and 10 ml of water. Another silicone column is washed with 5 ml of dichloromethane.

The synthesis subroutine includes eight steps. At 14, F-18 is provided. In detail, 0.2 ml of F-18/H218O is provided. The activity and dose are calculated.

At 15, the F-18 is filled in a labeled bottle 40 and heated on a heating plate so that the temperature of the F-18 reaches 95° C.

At 16, the loss of the water in the labeled bottle 40 is observed. When some of the water condenses on the column, there is vaporization in the labeled bottle 40.

At 17, 1.5 ml of the acetonitrile are filled in the labeled bottle 40 from the second bottle 31. The filling of the 1.5 ml of acetonitrile is done in three rounds separated from one another by a gap of 2 to 3 minutes, i.e., 0.5 ml in each round.

At 18, when there is almost no water in the labeled bottle 40, 2 ml of DMTEAN, a precursor of FDDNP, dissolved in 400 μl of acetonitrile are filled in the labeled bottle 40 from the sixth bottle 35. SN2 reaction is conducted at 95° C. for 15 minutes.

At 19, the reaction mixture in the labeled bottle 40 is cooled with helium for 2 minutes. Then, 15 ml of the 0.1N hydrochloride is filled in the labeled bottle 40 from the fifth bottle 34 for hydrolysis.

At 20, the product of the hydrolysis is directed through the C-column 50 from the labeled bottle 40. Then, the labeled bottle 40 is washed with 12 ml of the water from the third bottle 32.

At 21, 4 ml of the dichloromethane from the fourth bottle 33 are used to wash out the product from the C-18 column 50. The final product of F-18-FDDNP is collected in a collection bottle 60. Waste liquid produced in the process is collected in a waste bottle 70.

The radiochemistry purity of the F-18-FDDNP is analyzed with high pressure liquid chromatography (the “HPLC”). The HPLC uses a C-18 column with a size of 4.6×150. The flowing buffering liquid includes tetrahydrofuran, methanol and water at a ratio of 40:20:40. The flow rate is 0.5 ml/min. The wavelength of the ultraviolet light is 400 nm.

As discussed above, the F-18-FDDNP is produced in the automatic process according to the present invention. Therefore, operators are exposed to small doses of radiation. The average yield of the F-18-FDDNP is 41.19±5.23%. Analyzed with the HPLC under the conditions, the average radiochemistry purity is higher than the standard of 90%. Therefore, the automatic process according to the present invention is productive and simple.

The present invention has been described via the detailed illustration of the preferred embodiment. Those skilled in the art can derive variations from the preferred embodiment without departing from the scope of the present invention. Therefore, the preferred embodiment shall not limit the scope of the present invention defined in the claims.

1. An automatic process for synthesizing F-18-FDDNP comprising the steps of:
(A) filling potassium carbonate solution in a first bottle, acetonitrile in a second bottle, water in a third bottle, dichloromethane in a fourth bottle, 0.1N hydrochloride in a fifth bottle and a precursor, triflate, in a sixth bottle;
(B) washing each of the bottles with methanol;
(C) washing a C-18 column with 5 ml of ethanol and 10 ml of water, and washing another silicone column with 5 ml of dichloromethane;
(D) providing F-18, F-18/H218O, and calculating the activity and dose;
(E) filling the F-18 in a labeled bottle, and heating the F-18 on a heating plate so that the temperature of the F-18 reaches 95° C.;
(F) observing the loss of the water in the labeled bottle, wherein there is vaporization in the labeled bottle when some of the water condenses on the column;
(G) filling the acetonitrile in the labeled bottle from the second bottle in three rounds;
(H) filling DMTEAN, a precursor of FDDNP, dissolved in 400 μl of acetonitrile in the labeled bottle from the sixth bottle when there is almost no water in the labeled bottle for SN2 reaction;
(I) cooling the reaction mixture in the labeled bottle with helium, and filling the 0.1N hydrochloride in the labeled bottle from the fifth bottle for hydrolysis;
(J) directing the product of the hydrolysis through the C-18 column from the labeled bottle, and washing the labeled bottle with the water from the third bottle; and
(K) washing out the product from the C-18 column with the dichloromethane from the fourth bottle, and collecting the final product of F-18-FDDNP in a collection bottle.

2. The automatic process according to claim 1, wherein step (G) comprises the step of filling a total volume of 1.5 ml of the acetonitrile in the labeled bottle from the second bottle in three rounds separated from one another by a gap of 2 to 3 minutes.

3. The automatic process according to claim 1, wherein the SN2 reaction is conducted at 95° C. for 15 minutes at step (F).

4. The automatic process according to claim 1, wherein the average yield of the F-18-FDDNP is 41.19±5.23%, and the average radiochemistry purity is higher than 90%.
